SEQUENCE LISTING (4 sequences)

SEQ ID NO: 1 - protein, 381 aa, Mus musculus
MKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRK

SEQ ID NO: 2 - DNA, 1146 bp, Mus musculus
atgaaatggagtgataacaaagacgacattctcaaaggaggtgatgtggtgaggctcttccatgccgagcaagagaagtttctcacctgtgatgagcaccggaagaagcagcatgtgttcctgaggaccaccggcaggcagtcagccacgtcggccaccagttctaaagccctgtgggaagtggaggtagtccagcacgacccatgtcggggtggagctgggtactggaatagcctcttccggttcaagcacctggctacagggcattacttggctgcagaggtagaccctgactttgaggaagaatgcctggagtttcagccctcagtggaccctgatcaggatgcatctcggagtaggttgagaaacgcgcaagaaaaaatggtatactctctggtctccgtgcctgaaggcaacgacatctcctccatctttgagctagaccccacgactctgcgtggaggtgacagccttgtcccaaggaactcctatgtccgtctcagacacctgtgcaccaacacctgggtacacagcacaaacatccccatcgacaaggaagaggagaagcctgtgatgctgaaaattggtacctctcccctgaaggaggacaaggaagcatttgccatagttcctgtttcccctgctgaggttcgggacctggactttgccaatgatgccagcaaggtgctgggctccatcgctgggaagttggaaaagggcaccatcacccagaatgagagaaggtctgtcacgaagcttttggaagacttggtttactttgtcacgggtggaactaactctggccaagacgtgcttgaagttgtcttctctaagcccaatcgagagcggcagaagctgatgagggaacagaatattctcaagcagatcttcaagctgttgcaggcccccttcacggactgcggggatggcccgatgcttcggctggaggagctgggggatcagcgccatgctcctttcagacatatttgccgactctgctacagggtcctgcgacactcacagcaagactacaggaagaaccaggagtacatagccaagcagtttggcttcatgcagaagcagattggctatgacgtgctggccgaagacaccatcactgccctgctccacaacaaccggaaataa

SEQ ID NO: 3 - protein, 607 aa, Artificial
  IP3 Sponge Amino Acid sequence with GST-tag
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRK

SEQ ID NO: 4 - DNA, 1824 bp, Artificial
  IP3 sponge DNA sequence with GST tag
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgaaatggagtgataacaaagacgacattctcaaaggaggtgatgtggtgaggctcttccatgccgagcaagagaagtttctcacctgtgatgagcaccggaagaagcagcatgtgttcctgaggaccaccggcaggcagtcagccacgtcggccaccagttctaaagccctgtgggaagtggaggtagtccagcacgacccatgtcggggtggagctgggtactggaatagcctcttccggttcaagcacctggctacagggcattacttggctgcagaggtagaccctgactttgaggaagaatgcctggagtttcagccctcagtggaccctgatcaggatgcatctcggagtaggttgagaaacgcgcaagaaaaaatggtatactctctggtctccgtgcctgaaggcaacgacatctcctccatctttgagctagaccccacgactctgcgtggaggtgacagccttgtcccaaggaactcctatgtccgtctcagacacctgtgcaccaacacctgggtacacagcacaaacatccccatcgacaaggaagaggagaagcctgtgatgctgaaaattggtacctctcccctgaaggaggacaaggaagcatttgccatagttcctgtttcccctgctgaggttcgggacctggactttgccaatgatgccagcaaggtgctgggctccatcgctgggaagttggaaaagggcaccatcacccagaatgagagaaggtctgtcacgaagcttttggaagacttggtttactttgtcacgggtggaactaactctggccaagacgtgcttgaagttgtcttctctaagcccaatcgagagcggcagaagctgatgagggaacagaatattctcaagcagatcttcaagctgttgcaggcccccttcacggactgcggggatggcccgatgcttcggctggaggagctgggggatcagcgccatgctcctttcagacatatttgccgactctgctacagggtcctgcgacactcacagcaagactacaggaagaaccaggagtacatagccaagcagtttggcttcatgcagaagcagattggctatgacgtgctggccgaagacaccatcactgccctgctccacaacaaccggaaataa

(19) United States
(12) Patent Application Publication
(10) Pub. No.: US 2013/0296230 A1
(43) Pub. Date: Nov. 7, 2013

(54) METHOD FOR TREATING MALARIA, METHOD FOR KILLING MALARIA PARASITE, AND USE OF THE METHODS

(71) Applicants: Katsuhiko Mikoshiba, Wako-shi (JP); Masahiro Enomoto, Wako-shi (JP); Shin-Ichiro Kawazu, Obihiro-shi (JP)

(72) Inventors: Katsuhiko Mikoshiba, Wako-shi (JP); Masahiro Enomoto, Wako-shi (JP); Shin-Ichiro Kawazu, Obihiro-shi (JP)

(73) Assignee: RIKEN, Wako-shi, Saitama (JP)

(21) Appl. No.: 13/881,653

(22) PCT Filed: Oct. 27, 2011
(86) PCT No.: PCT/JP2011/074876

§ 371 Date: Jul. 15, 2013

Related U.S. Application Data
(60) Provisional application No. 61/407,679, filed on Oct. 28, 2010.

(51) Int. Cl. A61K 31/69; A61K 31/4045

(52) U.S. Cl. CPC A61K 31/69; A61K 31/4045
USPC 514/4.6; 558/298; 514/64; 548/506; 514/415; 514/44 R; 435/32; 435/29; 435/375

(57) ABSTRACT

A method of the present invention for treating malaria includes the step of administering, to a human or an animal, a therapeutically effective amount of drug for suppressing calcium ion exit from an intracellular organelle of the malaria parasite to an outside of the intracellular organelle and/or calcium ion entry from an outside of a cell of the malaria parasite into the cell.

### TECHNICAL FIELD

The present invention relates to a novel method for treating malaria, a novel method for killing a malaria parasite, and use of the methods.

### BACKGROUND ART

Malaria is one of protozoan diseases which is transmitted by an Anopheles mosquito, a kind of tropical mosquitoes. When an Anopheles mosquito sucks blood, malaria parasites living in the body of the Anopheles mosquito enter a human or animal body via a proboscis of the Anopheles mosquito.

Humans and animals are intermediate host for malaria parasites. Malaria parasites that have entered a human or animal body first accumulate in liver cells, where they proliferate. Then, the malaria parasites enter blood vessels and then invade erythrocytes, where they repeat further proliferation.

Some malaria treatment methods have been established already. For example, quinine, which is originally a naturally-occurring element, is known as a specific remedy for malaria. Currently, quinine can be produced by chemical synthesis. In addition, derivatives of quinine such as chloroquine and mefloquine are also known as therapeutic agents for malaria.

### CITATION LIST

### Non-Patent Literature 1

Flavio H. Beraldo, Katsuhiko Mikoshiba and Celia R. S. Garcia; J. Pineal Res. 2007, 43: p 360-364

### SUMMARY OF INVENTION

### Technical Problem

However, the quinine synthesis has a problem of insufficient yield, which has not been solved yet. Despite efforts of improving the quinine synthesis process made so far, the yield of net synthesis of quinine remains at single digit figures. Further, there is a problem that quinine has a very strong side-effect. Further, a problem of drug resistance is beginning to arise. Although chloroquine, which is a derivative of quinine, causes a lower side-effect as compared with quinine, a problem of drug resistance is beginning to arise as in the case of quinine. An artemisinin derivative is an anti-malaria drug that is most widely used currently. The artemisinin derivative is used in combination with an anti-malaria drug other than the artemisinin derivative in accordance with the recommendation of WHO in order to delay acquisition of resistance. If there occurs spread of a malaria parasite resistant to the artemisinin derivative in the future, there will be no alternative specific remedy. This is the largest problem now. In view of the above problems, development of a method and a drug for treating malaria that are based on a novel mechanism is earnestly desired.

Various approaches for investigating ecology of a malaria parasite that has entered a human or animal body in relation to melatonin are under study, regardless of whether these approaches lead to development of a method and a drug for treating malaria. For example, Non-Patent Literature 1 describes that human melatonin controls a circadian rhythm of a malaria parasite through activation of phospholipase C and that a melatonin-mediated signal transduction system is deeply involved with invasion, maturing, and proliferation of malaria parasites. More specifically, Non-Patent Literature 1 describes that an increase in concentration of calcium ion in a cytoplasm, which increase is brought about by melatonin, is suppressed by administering 2-aminoethyl diphenylborinate (2-APB), which is an InsP₃ (IP₃) receptor regulator, to malaria parasites. In addition, Non-Patent Literature 1 describes that 2-APB itself has no effect on promotion of calcium discharge in malaria parasites (see page 362, right column, the final line through page 363, left column, line 2, etc. of Non-Patent Literature 1).

However, Non-Patent Literature 1 merely describes an approach for unraveling a part of ecology of malaria parasites, and accomplished no result (e.g., killing of malaria parasites). As such, Non-Patent Literature 1 provides no suggestion about development of a method and a drug for treating malaria.

The present invention was accomplished in view of the above problems, and an object of the present invention is to provide a method for treating malaria, a method for killing a malaria parasite, each of which utilizes a mechanism different from a conventional one, and use of the methods.

### Solution to Problem

As a result of diligent studies for the purpose of solving the above problems, the inventors of the present invention found that an action mechanism of modulating calcium ion exit (discharge) from and/or calcium ion entry (influx) into a cytoplasm of a malaria parasite has a remarkably excellent effect on killing of the malaria parasite and on treatment of malaria. Based on this finding, the inventors of the present invention attained the present invention.

Specifically, a method of the present invention for treating malaria includes the step of administering, to a human or an animal, a therapeutically effective amount of drug for suppressing calcium ion exit from an intracellular organelle of a malaria parasite to an outside of the intracellular organelle and/or calcium ion entry from an outside of a cell of the malaria parasite into the cell.

A method of the present invention for killing a malaria parasite includes the step of supplying, to a malaria parasite, an effective amount of drug for suppressing calcium ion exit from an intracellular organelle of the malaria parasite to an outside of the intracellular organelle and/or calcium ion entry from an outside of a cell of the malaria parasite into the cell.

A malaria therapeutic agent of the present invention includes a drug for suppressing calcium ion exit from an intracellular organelle of a malaria parasite to an outside of the intracellular organelle and/or calcium ion entry from an outside of a cell of the malaria parasite into the cell.

A method of the present invention for screening for a candidate for a malaria therapeutic agent, includes: the first step of synchronized-culturing a malaria parasite in vitro and adding a drug to be screened while a developmental stage of the malaria parasite is being between a ring form stage and an early schizont stage; and the second step of selecting the drug as the candidate for the malaria therapeutic agent in a case where the addition of the drug suppresses development of the malaria parasite or kills the malaria parasite. A timing at which the drug to be screened is added is more preferably between a ring form stage and a trophozoite stage, further more preferably in an early ring form stage or a trophozoite stage, especially preferably in a trophozoite stage.

A method of the present invention for screening for a candidate for a malaria therapeutic agent, includes: the first step of adding a drug to be screened to a malaria parasite that is being cultured in vitro; the second step of measuring a first amount of calcium ions exited from an intracellular organelle of the malaria parasite to an outside of the intracellular organelle and/or a second amount of calcium ions entered from an outside of a cell of the malaria parasite into the cell; and the third step of selecting the drug as the candidate for the malaria therapeutic agent in a case where the addition of the drug reduces the first amount and/or the second amount.

A method of the present invention for preventing secondary infection with malaria includes the step of supplying, to blood that is outside a human or animal body and that is infected with a malaria parasite or has a risk of infection with the malaria parasite, a drug for suppressing calcium ion exit from an intracellular organelle of the malaria parasite to an outside of the intracellular organelle and/or calcium ion entry from an outside of a cell of the malaria parasite into the cell.

### Advantageous Effects of Invention

The present invention can provide a method for treating malaria, a method for killing a malaria parasite, each of which utilizes a mechanism different from a conventional one, and use of the methods.

### BRIEF DESCRIPTION OF DRAWINGS

FIG. 1

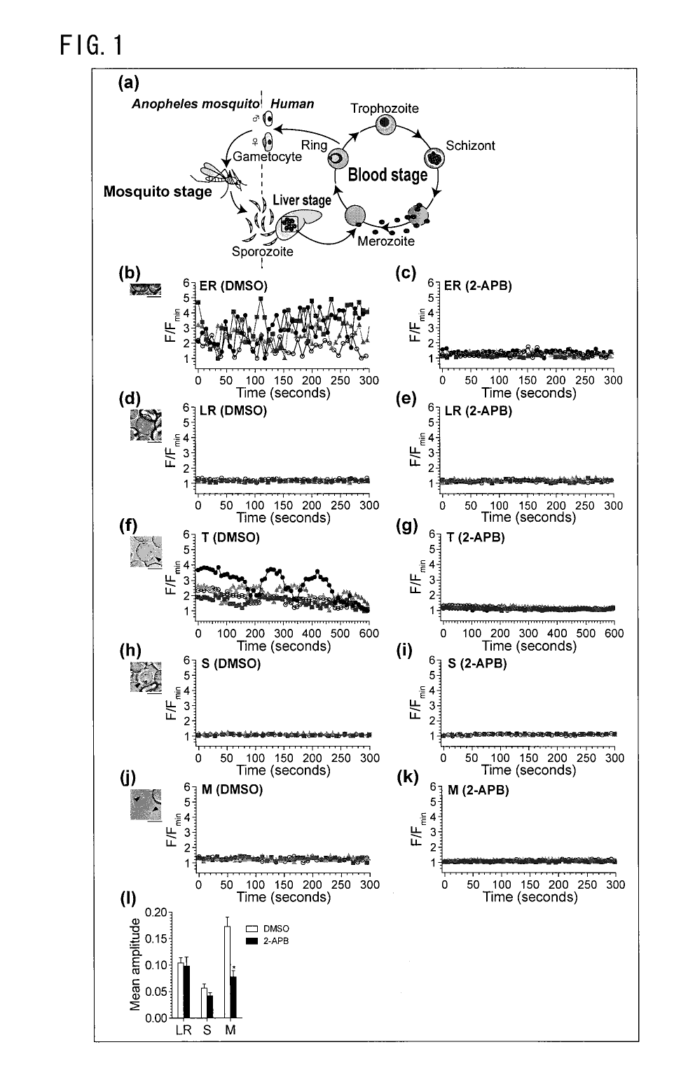

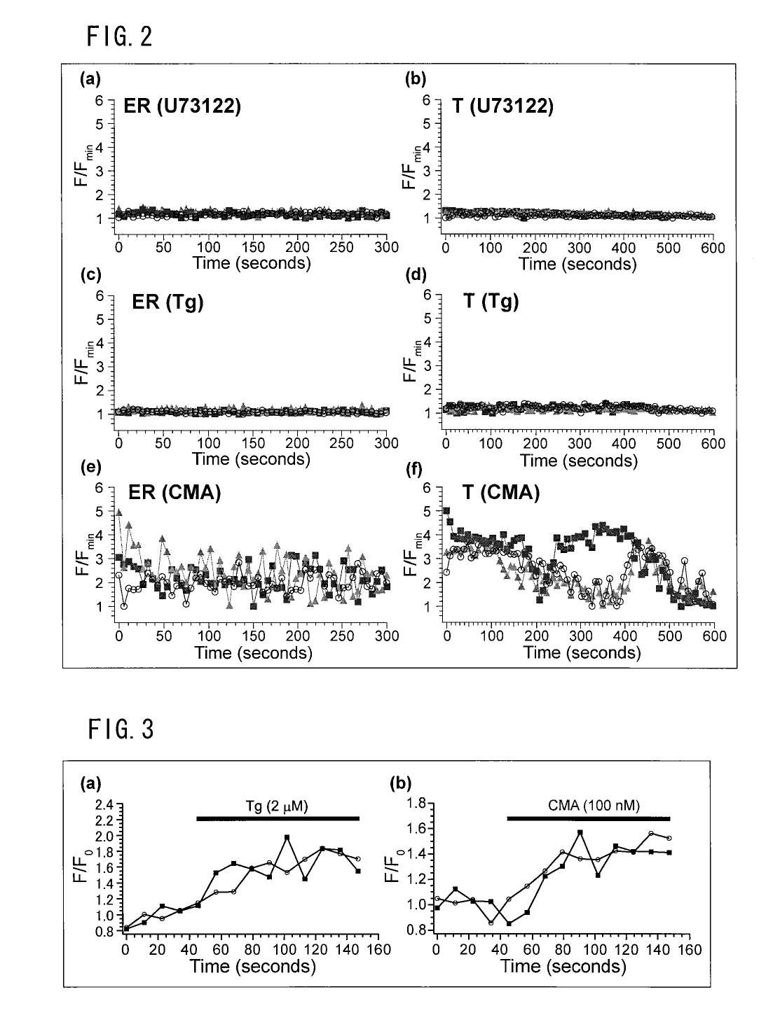

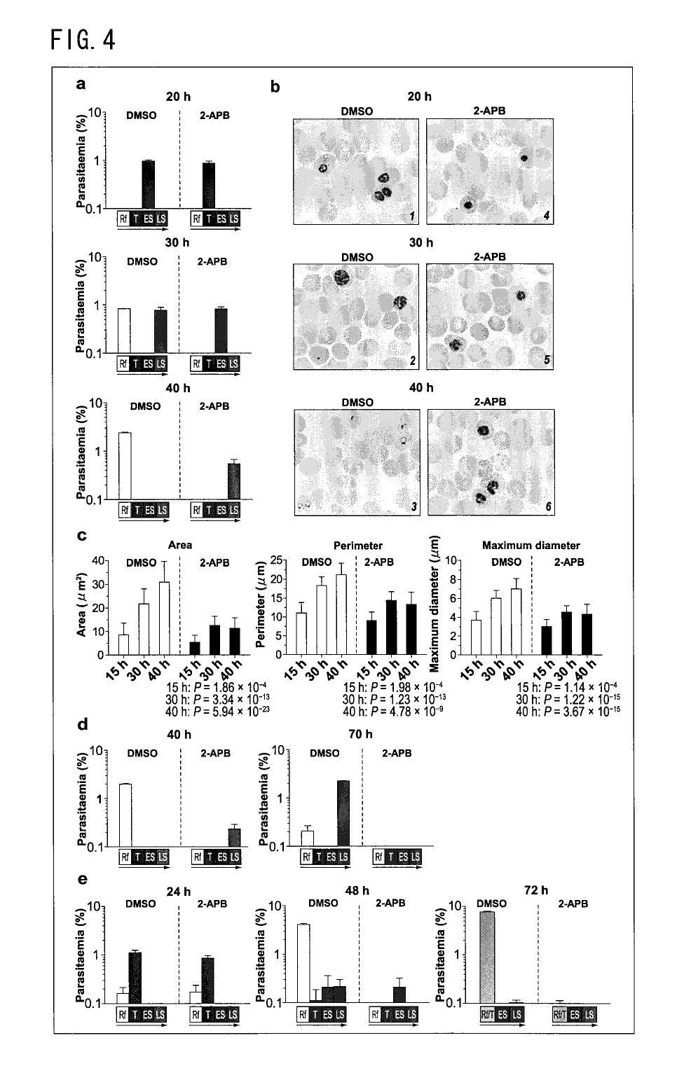

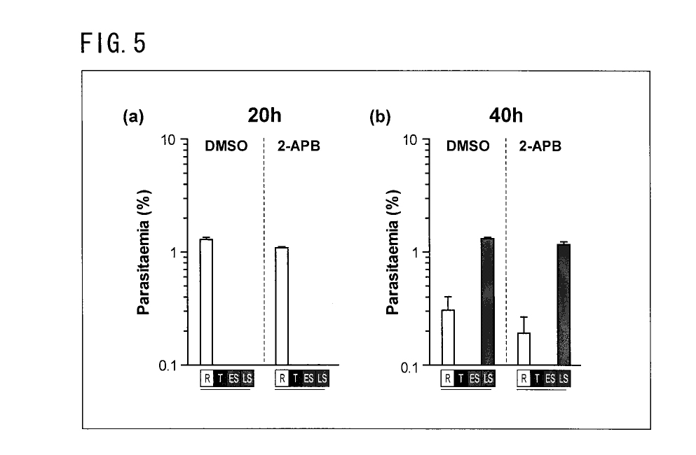

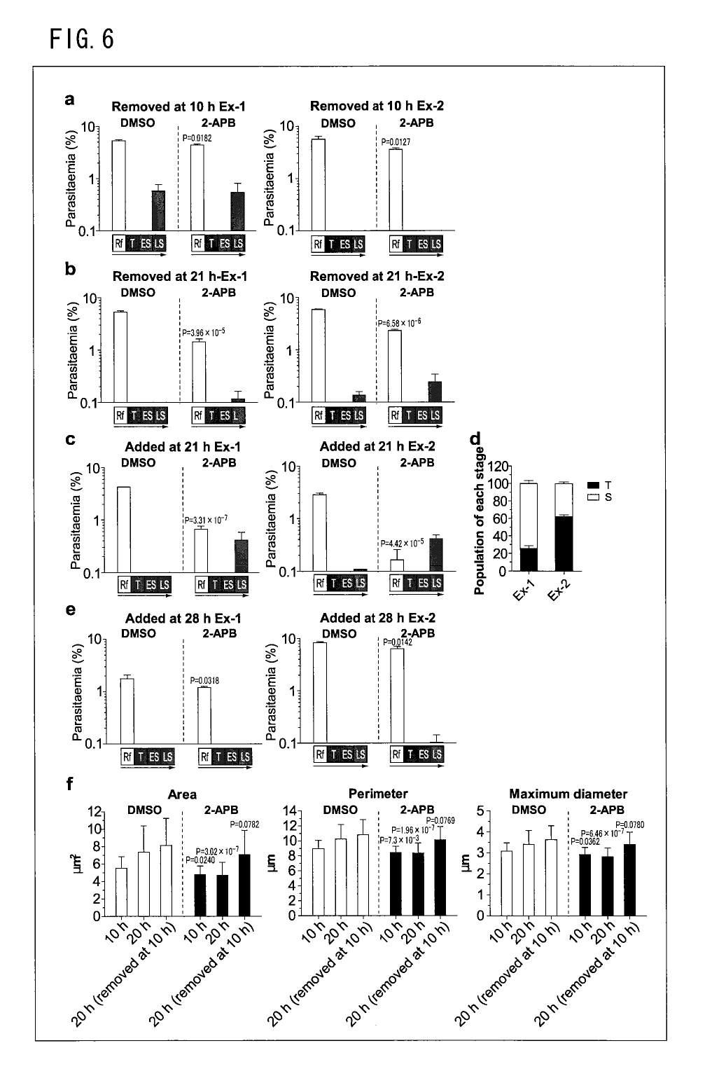

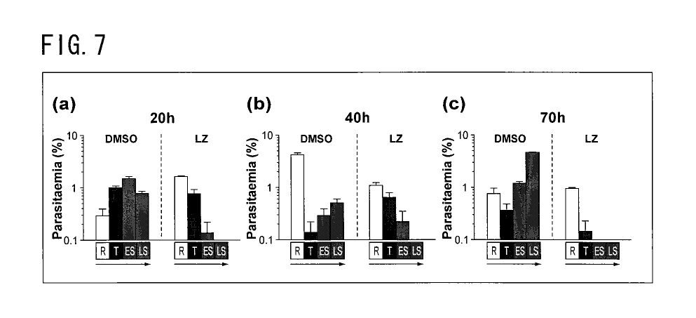

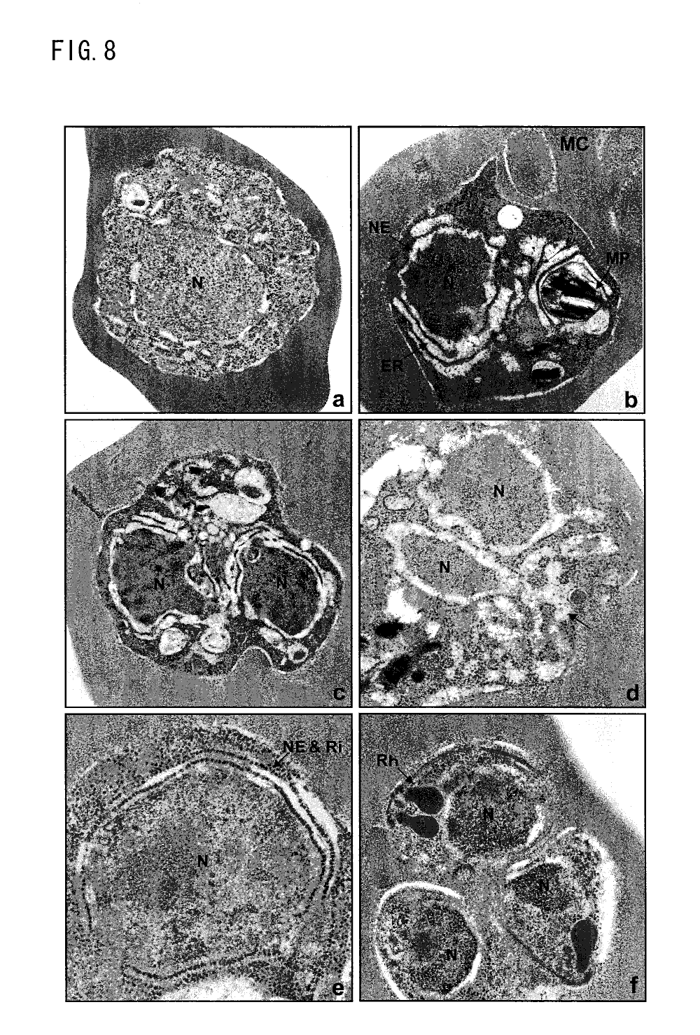

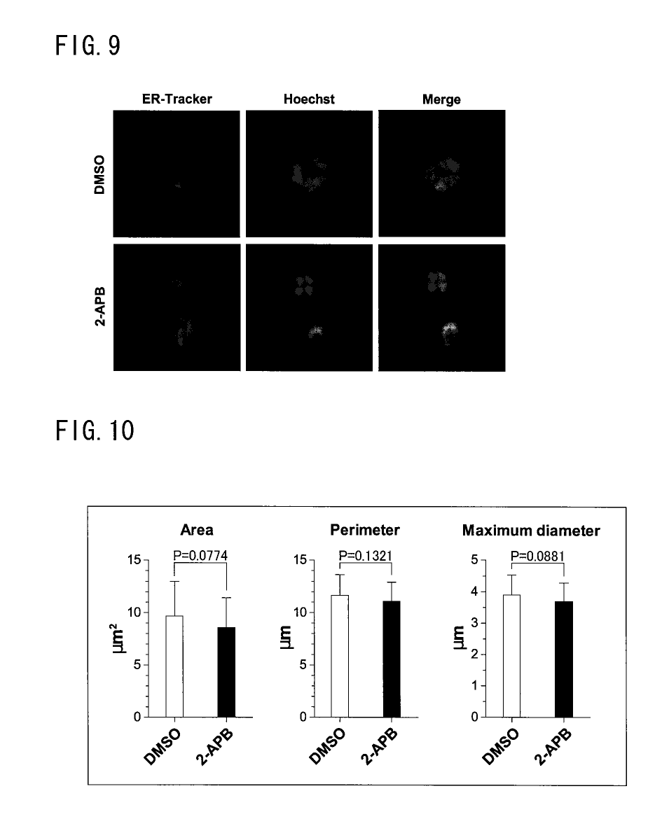

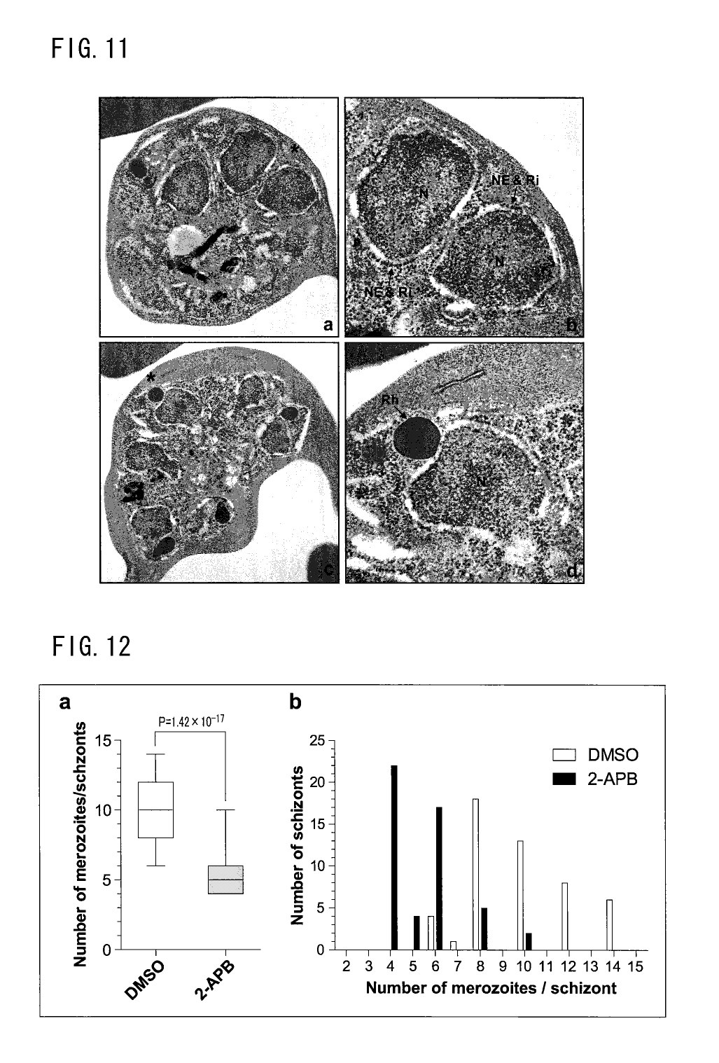

(a) of FIG. 1 shows a life cycle of a falciparum malaria parasite, mainly focusing on an erythrocyte parasitic stage.

(b) through (k) of FIG. 1 are graphs showing results of intracellular fluorescence Ca²⁺ imaging of falciparum malaria parasites in stages from an early ring form stage to a schizont stage. The photographs in FIG. 1 show images of intraerythrocytic malaria parasite in respective stages into which a fluorescence Ca²⁺ indicator was introduced. (1) of FIG. 1 shows a result of an analysis of an effect of 2-APB on an amplitude of periodic Ca²⁺ fluctuations in late ring form, schizont and merozoite stages.

FIG. 2

(a) and (b) of FIG. 2 are graphs each showing a result of intracellular fluorescence Ca²⁺ imaging of U73122-treated falciparum malaria parasites in early ring form and trophozoite stages. (c) and (d) of FIG. 2 are graphs each showing a result of intracellular fluorescence Ca²⁺ imaging of thapsigargin-treated falciparum malaria parasites in early ring form and trophozoite stages. (e) and (f) of FIG. 2 are graphs each showing a result of intracellular fluorescence Ca²⁺ imaging of concanamycin A-treated falciparum malaria parasites in early ring form and trophozoite stages.

FIG. 3

(a) and (b) of FIG. 3 are graphs showing results of intracellular fluorescence Ca²⁺ imaging of thapsigargin-treated falciparum malaria parasites and concanamycin A-treated falciparum malaria parasites, respectively, in early ring form and trophozoite stages with the use of perfusion experiments.

FIG. 4

FIG. 4 shows results of experiments on inhibition of intraerythrocytic falciparum malaria parasite development by 2-APB. (a) of FIG. 4 shows a result of 40 hours of synchronized culturing, (b) of FIG. 4 shows morphology of falciparum malaria parasites synchronized-cultured for 40 hours, (c) of FIG. 4 shows a result of analysis for area, perimeter and maximum diameter of malaria parasite cells, (d) of FIG. 4 shows a result of 70 hours of synchronized culturing, and (e) of FIG. 4 shows a result of 2-APB treatment of a chloroquine-resistant strain of falciparum malaria parasites.

FIG. 5

(a) and (b) of FIG. 5 show results at 20 hours and 40 hours from start of synchronized-culturing of falciparum malaria parasites with the use of 2-APB-pretreated erythrocytes.

FIG. 6

FIG. 6 shows results of experiments on an effect of 2-APB on cultures of falciparum malaria parasites in different developmental stages. In (a) of FIG. 6, 2-APB was added at start of culturing, and culture medium was replaced with culture medium containing no 2-APB at 10 hours from start of the culturing. In (b) of FIG. 6, 2-APB was added at start of culturing, and culture medium was replaced with culture medium containing no 2-APB at 21 hours from start of the culturing. In (c) of FIG. 6, 2-APB was added at 21 hours from start of the culturing. In (e) of FIG. 6, 2-APB was added at 28 hours from start of the culturing. (a) through (c) of FIG. 6 each show a result at 40 hours from start of culturing, and (e) of FIG. 6 shows a result at 45 hours from start of culturing. (f) of FIG. 6 shows a result of analysis of an effect of 100 μM 2-APB on area, perimeter and maximum diameter of malaria parasites cells.

FIG. 7

FIG. 7 shows results of experiments on inhibition of intraerythrocytic falciparum malaria parasite development by luzindole (LZ). (a) of FIG. 7 shows a result of 20-hour synchronized culturing, (b) of FIG. 7 shows a result of 40-hour synchronized culturing, and (c) of FIG. 7 shows a result of 70-hour synchronized culturing.

FIG. 8

FIG. 8 shows electron micrographs of controls and falciparum malaria parasites treated with 2-APB. (a) of FIG. 8 shows an electron micrograph of a falciparum malaria parasite cultured for 30 hours after DMSO administration (control group; original magnification: ×30,000). (b) of FIG. 8 shows an electron micrograph of a falciparum malaria parasite cultured for 30 hours after 100 μM 2-APB administration (original magnification: ×30,000). (c) of FIG. 8 shows an electron micrograph of a falciparum malaria parasite cultured for 30 hours after 100 μM 2-APB administration (original magnification: ×30,000). (d) of FIG. 8 shows an electron micrograph of a falciparum malaria parasite cultured for 30 hours after 100 μM 2-APB administration. In the electron micrograph of (d) of FIG. 8, a reticular endoplasmic reticulum structure (indicated by the arrow) is observed (original magnification: ×50,000). (e) of FIG. 8 shows an electron micrograph of a falciparum malaria parasite cultured for 40 hours after 100 μM 2-APB administration. In the electron micrograph of (e) of FIG. 8, a structure (indicated by the arrow) in which a nuclear envelope (NE) is surrounded by ribosomal granules (Ri) is observed (original magnification: ×50,000). (f) of FIG. 8 shows an electron micrograph of a falciparum malaria parasite cultured for 40 hours after 100 μM 2-APB administration. In the electron micrograph of (f) of FIG. 8, formation of rhoptries (Rh) and other micro-organelles is observed (original magnification: ×50,000). In FIG. 8, N represents a nucleus, MP represents a malaria pigment, and MC represents a Maurer's cleft.

FIG. 9

FIG. 9 shows an effect of 2-APB on an endoplasmic reticulum structure. Specifically, FIG. 9 shows a falciparum malaria parasite nucleus and endoplasmic reticulum that were stained simultaneously with Hoechst 33342 (blue) and ER-Tracker (red) after 30 hours of culturing in the presence of DMSO (upper panels) or 2-APB (lower panels). Merged images of a result of the staining with Hoechst 33342 (blue) and a result of the staining with ER-Tracker (red) are shown in the right columns (Merge).

FIG. 10

FIG. 10 shows a result of observation of area, perimeter and maximum diameter of malaria parasite cells in (i) a group in which a chloroquine-resistant strain of malaria parasites infecting erythrocytes were synchronized-cultured for 24 hours in the presence of 100 μM DMSO and (ii) a group in which a chloroquine-resistant strain of malaria parasites infecting erythrocytes were synchronized-cultured for 24 hours in the presence of 100 μM 2-APB. The error bars represent mean±S.D. (n=50). P values are given in each panel (two-tailed unpaired t test).

FIG. 11

FIG. 11 shows electron micrographs of malaria parasites in a DMSO control culture. (a) of FIG. 11 shows an electron micrograph obtained at 30 hour from start of an assay (original magnification: ×30,000). (b) of FIG. 11 shows the part indicated by the asterisk in (a) of FIG. 11 at a higher magnification (original magnification: ×80,000). (c) of FIG. 11 shows an electron micrograph obtained at 30 hour from start of an assay (original magnification: ×20,000). (d) of FIG. 11 shows the part indicated by the asterisk in (c) of FIG. 11 at a higher magnification (original magnification: ×80,000). In FIG. 11, N represents a nucleus, NE represents a nuclear envelope, Ri represents a ribosome, and Rh represents a rhoptry.

FIG. 12

FIG. 12 shows an effect of 2-APB on the number of merozoites in each schizont. (a) of FIG. 12 shows, by box plots, the number of merozoites (M) formed in each schizont (S) in a case where falciparum malaria parasites were cultured for 40 hours in the presence of DMSO (indicated by the white box) or 2-APB (indicated by the shaded box). The rectangles located in the middle correspond to a range from a first quantile to a third quantile, the segment in each of the rectangles indicates a median, and the upper and lower lines extending from each of the boxes indicate a maximum value and a minimum value, respectively. (b) of FIG. 12 is a histogram of the data shown in (a) of FIG. 12.

### DESCRIPTION OF EMBODIMENTS

An embodiment of the present invention is described below in detail.

[1. Method for Treating Malaria]

(Outline of Method for Treating Malaria)

A method of the present invention for treating malaria includes the step of administering, to a human or an animal, a therapeutically effective amount of drug (hereinafter referred to as “Ca transport inhibitor”) for suppressing calcium ion exit from an intracellular organelle of a malaria parasite to an outside of the intracellular organelle and/or calcium ion entry from an outside of a cell of the malaria parasite into the cell.

The method of the present invention for treating malaria is a novel treatment method utilizing a mechanism utterly different from that used for a conventional malaria therapeutic agent. Therefore, the method of the present invention for treating malaria is effective also for treatment of infection with a malaria parasite which exhibits resistance to conventional malaria therapeutic agents such as quinine and chloroquine. Further, the Ca transport inhibitor used as an active ingredient is selectable also from compounds that are easy to synthesize. In such a case, low-cost and mass supply of the Ca transport inhibitor is possible. Furthermore, the Ca transport inhibitor has an advantage of being selectable from compounds that bring about a relatively weak side-effect on a human or an animal to be treated.

It is estimated that the active calcium ion exit or entry in a malaria parasite is mainly carried out by receptors that are collectively referred to as calcium ion channel linked-receptors. The inventors of the present invention separately administered, to malaria parasites, (i) a human melatonin receptor inhibitor, (ii) a human inositol trisphosphate receptor inhibitor, and (iii) a compound that has a binding activity specific to a inositol trisphosphate (inositol 1,4,5-trisphosphate), and as a result, the malaria parasites were killed in each of these cases (see Examples that will be described later). Based on this fact, the inventors of the present invention attained the present invention. These three types of inhibitors etc. inhibit a series of inositol trisphosphate receptor-mediated signal transduction systems in a human and modulate discharge or influx of a calcium ion from/into cells. Further, the inventors of the present invention found that the drug is preferably one that inhibits especially calcium ion exit from an intracellular organelle of a malaria parasite in a ring form and/or a trophozoite to an outside of the intracellular organelle and/or calcium ion entry from an outside of a cell of the malaria parasite into the cell. This suggests that a malaria parasite also has a similar signal transduction system to that of a human.

Especially the fact that creating an inositol-trisphosphate-depletion condition with the use of a human inositol trisphosphate receptor inhibitor or a compound (e.g., peptide) having a binding activity specific to inositol trisphosphate killed the malaria parasites suggests that the malaria parasites have an inositol trisphosphate receptor. Note that it has not been known that a sequence which shows homology with a human inositol trisphosphate receptor is present in the genome sequence of the malaria parasite.

As for humans, it is known that (i) the inositol trisphosphate receptor is present not only in an endoplasmic reticulum, which is one of intracellular organelles, but also in a nucleus and a cell membrane and (ii) the inositol trisphosphate receptor contributes to active calcium ion entry from an outside of a cell into the cell or active calcium ion exit from an intracellular organelle to an outside of the intracellular organelle. Above all, especially calcium ion exit from an endoplasmic reticulum, which is one of intracellular organelles, to an outside of the endoplasmic reticulum (into a cytoplasm) is a function of high importance for the present invention. Further, the drug is preferably one that inhibits especially calcium ion exit from an intracellular organelle of a malaria parasite in a ring form and/or a trophozoite to an outside of the intracellular organelle.

(About Malaria Treatment)

“Malaria”, which is a target of treatment in the present invention, refers to, in a broad sense, a state where a human or an animal is infected with a malaria parasite belonging to the genus Plasmodium. “Malaria” is a concept encompassing not only a state where a human or an animal is developing a symptom characteristic to malaria, but also a state where the human or the animal has not expressed such a symptom yet. Examples of the symptom specific to malaria encompass, but are not limited to, headache, a feeling of weariness, anemia, splenomegaly, and fever-related seizure including a cold stage, a hot stage, and a sweat stage.

“Treatment” in the present invention refers to suppressing activity of a malaria parasite in a human or an animal as compared with a case where no measure is taken, and preferably refers to killing the malaria parasite. One aspect of the treatment encompasses reducing or alleviating at least one of the symptoms associated with malaria, for example, reducing or alleviating headache, a feeling of weariness, anemia, splenomegaly, and fever-related seizure including a cold stage, a hot stage, and a sweat stage.

(Human or Animal to be Treated)

A subject to be treated is a human or an animal who/which is infected with a malaria parasite, more specifically any one selected from the group consisting of reptiles, birds, and mammals including humans, each of which is known as a host for a malaria parasite. Above all, the method of the present invention for treating malaria is suitably applied especially to mammals. A kind of a mammal to be treated is not limited in particular. Examples of the mammal to be treated encompass laboratory animals such as mice, rats, rabbits, guinea pigs, and primates except for humans; pet animals (pets) such as dogs and cats; farm animals such as cattle and horses; and humans. Above all, humans are especially preferable.

A route via which a human or an animal is infected with a malaria parasite is not limited in particular. Examples of such a route encompass infections via bites of mosquitos belonging to the genus Anopheles, infections via transfusion of blood (infected blood) including a malaria parasite, and infections from mothers to infants through placenta, and infections via injection needles.

(Kind and Developmental Stage of Malaria Parasite)

A kind of a malaria parasite which is a target of the treatment and to be killed is not limited in particular,

provided that it can infect a human or an animal. Representative examples of a malaria parasite which can infect a human encompass five kinds of malaria parasites, i.e., falciparum malaria parasites (P. falciparum), quartan malaria parasites (P. malariae), vivax malaria parasites (P. vivax), oval malaria parasites (P. ovale), and P. knowlesi. In addition, malaria parasites, such as P. cynomolgi, which are known as monkey malaria parasites are important as malaria parasites that are highly likely to be reported in the future as having infected humans.

A developmental stage of a malaria parasite which is a target of the treatment and to be killed is not limited in particular. In order to maximize treatment and killing effects, it is sometimes preferable to treat and kill a malaria parasite, in an erythrocyte, whose developmental stage is between a ring form and an early schizont, it is sometimes more preferable to treat and kill a malaria parasite, in an erythrocyte, whose developmental stage is between a ring form and a trophozoite, it is sometimes especially preferable to treat and kill a malaria parasite, in an erythrocyte, whose developmental stage is an early ring form or a trophozoite, and it is sometimes most preferable to treat and kill a malaria parasite, in an erythrocyte, whose developmental stage is a trophozoite.

(Ca Transport Inhibitor as Active Ingredient)

In the method of the present invention for treating malaria, a Ca transport inhibitor is used as an active ingredient for malaria treatment. The Ca transport inhibitor is not limited to a specific one, provided that it has a function of suppressing calcium ion exit from an intracellular organelle of a malaria parasite to an outside of the intracellular organelle and/or calcium ion entry from an outside of a cell of the malaria parasite into the cell. The Ca transport inhibitor is preferably selected from compounds that have a function of suppressing active calcium ion exit from an intracellular organelle of a malaria parasite to an outside of the intracellular organelle, more preferably selected from compounds that have a function of inhibiting especially calcium ion exit from an intracellular organelle of a malaria parasite in a ring form and/or a trophozoite to an outside of the intracellular organelle. What is intended as “intracellular organelle” is, for example, an endoplasmic reticulum of the malaria parasite.

Examples of the compound that has a function of suppressing active calcium ion exit from an intracellular organelle of a malaria parasite to an outside of the intracellular organelle encompass (1) an inhibitor of melatonin, an inhibitor of a melatonin homolog in a malaria parasite, an inhibitor of melatonin receptor, or an inhibitor of a melatonin receptor homolog in a malaria parasite, (2) an inhibitor of an inositol trisphosphate receptor or an inhibitor of an inositol trisphosphate receptor homolog in a malaria parasite, and (3) a compound and a peptide that have a binding activity specific to inositol trisphosphate, and a nucleic acid encoding the peptide. Each of these inhibitors etc. in (1) through (3) has a function of suppressing inositol trisphosphate-induced calcium release (discharge).

What is meant by “inositol trisphosphate-induced calcium release” is a physiological phenomenon in which melatonin or a homolog thereof induces production of inositol trisphosphate in a living body and then the inositol trisphosphate triggers release of a calcium ion (bivalent). In general, inositol trisphosphate induces release of a calcium ion by binding to an inositol trisphosphate receptor, which functions as a calcium ion channel linked-receptor, or to a homolog thereof.

The inhibitor of melatonin, the inhibitor of a melatonin homolog in a malaria parasite, the inhibitor of melatonin receptor, and the inhibitor of a melatonin receptor homolog in a malaria parasite are not each limited to a specific one, provided that they prevent melatonin or a homolog thereof from binding to a corresponding receptor (a melatonin receptor or a homolog of a melatonin receptor) so as to block or suppress signal transduction towards downstream.

The inhibitor of a melatonin receptor or the inhibitor of a melatonin receptor homolog in a malaria parasite is not limited to a specific one, and can be, for example, a human melatonin receptor modulator or antagonist; or a serotonin agonist or antagonist which is a physiologically active substance having a similar structure to melatonin. Above all, luzindole, 4P-ADOT, 4P-PDOT, and the like are suitably used.

The inhibitor of melatonin or the inhibitor of a melatonin homolog in a malaria parasite is not limited to a specific one, and can be, for example, a compound or a peptide which has a binding activity specific to melatonin or a homolog thereof, or a nucleic acid encoding the peptide. An example of the peptide which has a binding activity specific to melatonin or a homolog thereof is a peptide that constitutes a melatonin (or a melatonin homolog) binding domain of a melatonin receptor or a homolog thereof.

When simply using the term “melatonin” or “melatonin receptor”, what is intended as an animal kind (including an human) from which the melatonin or the melatonin receptor is derived is any one of all the animal species except for malaria parasites, especially a human or an animal to be treated. Further, what is intended as a melatonin homolog or a melatonin receptor homolog is one derived from a malaria parasite.

The inhibitor of a inositol trisphosphate receptor or the inhibitor of an inositol trisphosphate receptor homolog in a malaria parasite is not limited to a specific one, and can be, for example, any of the compounds (human inositol trisphosphate receptor modulators and antagonists) disclosed in Japanese Patent Application Publication, Tokukai, No. 2007-169272 A which is incorporated herein by reference. Above all, 2-aminoethyl diphenylborinate (2-APB), heparin, Xestospongin C, etc. are more preferable. Each of these inhibitors prevents inositol trisphosphate from binding to a corresponding receptor (an inositol trisphosphate receptor or a homolog thereof) so as to block or suppress signal transduction towards downstream.

The compound or peptide that has a binding activity specific to inositol trisphosphate, and the nucleic acid encoding the peptide are not limited to a specific one, and can be, for example, a polypeptide that has a high affinity with inositol trisphosphate and a nucleic acid encoding the peptide, which are disclosed in U.S. Pat. No. 6,465,211 and U.S. Pat. No. 7,041,440 (Japanese Patent Application Publication, Tokukai, No. 2000-135095 A) that are incorporated herein by reference. Above all, a peptide consisting of the amino acid sequence represented by SEQ ID NO: 1 and those containing a nucleic acid which encodes the peptide and which has the nucleotide sequence represented by SEQ ID NO: 2 are more preferable. The peptide may be a peptide that has 80% or more sequence homology, preferably 90% or more sequence homology, especially preferably 95% or more sequence homology with the peptide consisting of the amino acid sequence represented by SEQ ID NO: 1, as long as the function of having the binding activity specific to inositol trisphosphate is not impaired. A person skilled in the art can appropriately use a peptide that is encoded by a nucleotide sequence that hybridizes, under a stringent condition, to a sequence complementary to a nucleotide sequence encoding the aforementioned peptide or to a probe that can be prepared from the nucleotide sequence encoding the aforementioned peptide. The stringent condition is, for example, washing once, preferably two to three times at 60° C. under a salt concentration corresponding to a 1×SSC buffer containing 0.1% SDS, preferably a 0.1×SSC buffer containing 0.1% SDS. Each of these compounds and peptides prevents inositol trisphosphate from binding to a corresponding receptor (an inositol trisphosphate receptor or a homolog thereof) so as to block or suppress signal transduction towards downstream.

Expression of the peptide in a mammal cell can be more stabilized by giving a GST tag to an N-terminus side of the peptide, for example. A peptide consisting of the amino acid sequence represented by SEQ ID NO: 3 corresponds to a sequence obtained by giving a linker sequence and a GST tag to an N-terminus side of the peptide consisting of the amino acid sequence represented by SEQ ID NO: 1. A nucleic acid having the nucleotide sequence represented by SEQ ID NO: 4 encodes the peptide consisting of the amino acid sequence represented by SEQ ID NO: 3.

When simply using the term “inositol trisphosphate receptor”, what is intended as an animal species (including humans) from which the inositol trisphosphate receptor is derived is all the animal kinds except for malaria parasites, especially a human or an animal to be treated. What is intended as the inositol trisphosphate receptor homolog is one derived from a malaria parasite.

From the perspective of obtaining a more direct and effective treatment effect, it is sometimes preferable to block or suppress signal transduction at a more downstream side of a signal transduction system to which melatonin and inositol trisphosphate are involved. That is, it is sometimes preferable to use an inhibitor of an inositol trisphosphate receptor or an inhibitor of an inositol trisphosphate receptor homolog in a malaria parasite.

From the perspective of giving no substantial influence on a human or an animal to be treated, it is sometimes preferable to select the Ca transport inhibitor from (i) an inhibitor that specifically acts on a melatonin homolog in a malaria parasite, (ii) an inhibitor that specifically acts on a melatonin receptor homolog in a malaria parasite, and (iii) an inhibitor that specifically acts on an inositol trisphosphate receptor homolog in a malaria parasite. Note, however, that even in a case where the signal transduction system to which melatonin and inositol trisphosphate are involved is temporarily blocked or suppressed, no fatal influence is caused on the human or the animal.

(Administration Method and Dose)

The method of the present invention for treating malaria includes the step of administering a therapeutically effective amount of at least one kind of the Ca transport inhibitors to a human or an animal infected with a malaria parasite. The Ca transport inhibitor may be administered alone or may be administered as one constituent of a pharmaceutical composition suitable for an objective of the administration.

A method of administering the Ca transport inhibitor is not limited in particular. The Ca transport inhibitor may be systemically administered by a method such as oral administration, intravascular administration (intravenous administration or intraarterial administration), or enteral administration or may be locally administered by a method such as transdermal administration or sublingual administration. According to one preferable administration, the Ca transport inhibitor is systemically administered intravenously or intraarterially in order that the Ca transport inhibitor may act on a malaria parasite living in a blood vascular system (within an erythrocyte). According to another preferable administration, the Ca transport inhibitor is administered orally, from the viewpoint of easiness of administration, etc.

A dose (therapeutically effective amount) of the Ca transport inhibitor can be appropriately determined in accordance with age, sex, symptom of the human or the animal into which the Ca transport inhibitor is to be administered, administration route, the number of administrations, etc. If necessary, an in vivo assay using the Ca transport inhibitor can be carried out in advance. This makes it possible to determine the dose of the Ca transport inhibitor without the need for excessive number of experiments.

For example, in a case where the Ca transport inhibitor is a so-called low-molecular compound, a preferable example of the dose is in a range of not less than 0.5 mg and not more than 20 mg, in a range of not less than 0.5 mg and not more than 10 mg, or in a range of not less than 1 mg and not more than 5 mg per kilogram of the weight of the human or the animal.

The number of administrations of the Ca transport inhibitor is not limited in particular, provided that a treatment effect can be obtained. For example, the number of administrations of the Ca transport inhibitor can be appropriately determined in accordance with the kind, dose, administration route of the Ca transport inhibitor, symptom, age, and sex of the human or the animal.

A timing at which the Ca transport inhibitor is administered is not limited in particular, provided that a treatment effect can be obtained. However, in order to maximize the treatment effect, it is sometimes preferable to determine the timing in accordance with a developmental stage of the malaria parasite. More specifically, it is sometimes preferable to determine the timing at which the Ca transport inhibitor is administered so that a blood concentration of the Ca transport inhibitor reaches the therapeutically effective amount in a state in which the malaria parasite whose developmental stage is between a ring form and an early schizont is present in an erythrocyte of the human or the animal. More preferably, the timing at which the Ca transport inhibitor is administered is determined so that a blood concentration of the Ca transport inhibitor reaches the therapeutically effective amount in a state in which the developmental stage of the malaria parasite is between a ring form and a trophozoite. Especially preferably, the timing at which the Ca transport inhibitor is administered is determined so that a blood concentration of the Ca transport inhibitor reaches the therapeutically effective amount in a state in which the developmental stage of the malaria parasite is an early ring form or a trophozoite. Most preferably, the timing at which the Ca transport inhibitor is administered is determined so that a blood concentration of the Ca transport inhibitor reaches the therapeutically effective amount in a state in which the developmental stage of the malaria parasite is a trophozoite.

Note that the scope of the method of the present invention for treating malaria encompasses so-called preventive administration, i.e., administration of the Ca transport inhibitor before the human or the animal is infected with the malaria parasite. That is to say, a state in which the blood concentration of the Ca transport inhibitor is kept equal to or higher than the therapeutically effective amount is created before the human or the animal is infected with the malaria parasite, so that a treatment effect is brought about when the human or the animal is infected with the malaria parasite.

Developmental stages of malaria parasites parasitizing the body of the human or the animal may be synchronized at the same time as or prior to the administration of the drug. The synchronization of developmental stages can be carried out, for example, by administering melatonin into the body of the human or the animal.

Note that a person skilled in the art can easily grasp the developmental stage of the malaria parasite in the human or the animal. One example of a method for grasping the developmental stage is a method of preparing a thin smear of an erythrocyte, staining the thin smear by a method such as Giemsa staining, and then observing the malaria parasite by a microscope. In a case where periodicity is seen in malaria parasite development, even a developmental stage after elapse of a predetermined period of time can be estimated once the developmental stage is checked.

Further, by carrying out an in vivo assay or the like as needed, a person skilled in the art can easily grasp the blood concentration of the Ca transport inhibitor in the human or the animal, more specifically, a relationship among the dose, administration timing, and blood concentration of the Ca transport inhibitor.

(Combination Therapy)

The method of the present invention for treating malaria may be combined with a malaria treatment method, other than the method of the present invention for treating malaria, such as a method using quinine, chloroquine, mefloquine, or an artemisinin derivative (combination treatment). The method of the present invention for treating malaria is a novel treatment method utilizing a mechanism different from any of the conventional malaria therapeutic agents. Accordingly, use of the combination treatment can be expected to produce a synergistic effect with a conventional treatment method and dramatically improve a treatment outcome. Further, use of the method of the present invention for treating malaria in combination with an artemisinin derivative can be expected to produce an effect of delaying acquisition of resistance by the malaria parasite.

[2. Method for Killing Malaria]

A method of the present invention for killing a malaria parasite includes the step of supplying an “effective amount” of the Ca transport inhibitor to the malaria parasite.

What is intended by “effective amount” is an amount which makes it possible to kill the malaria parasite, and is appropriately determined by a person skilled in the art in accordance with a condition such as an environment in which the malaria parasite to which the drug is to be administered is living.

An example of application of the method for killing a malaria parasite is suppressing a risk of secondary infection with malaria by supplying the Ca transport inhibitor to blood that has turned out to be infected with a malaria parasite or has a risk of infection with a malaria parasite. Such blood is not limited in particular, and encompasses also blood taken out of human or animal bodies, such as blood collected by blood donation campaigns, blood for transfusions, blood shed by outdoor activities (including traffic accidents etc.), and blood shed by medical activities.

An example of a method for checking an effect of killing a malaria parasite is a method of preparing a thin smear of an erythrocyte, staining the thin smear by a method such as Giemsa staining, and then observing the malaria parasite microscopically.

The descriptions in [1. Method for Treating Malaria] can be referred to as for the kind, developmental stage, etc. of the malaria parasite to which the method of the present invention for killing a malaria parasite is applied.

[3. Malaria Therapeutic Agent]

A malaria therapeutic agent of the present invention includes the Ca transport inhibitor.

The malaria therapeutic agent may be constituted only by the Ca transport inhibitor or may be a pharmaceutical composition containing the Ca transport inhibitor as one constituent.

Other components than the Ca transport inhibitor that constitute the pharmaceutical composition are not limited in particular. The Ca transport inhibitor may be used in combination with, for example, a carrier, a lubricant, a preservative, a stabilizer, a humectant, an emulsifier, salts for osmotic adjustment, a buffer, a colorant, a flavoring, a sweetener, an antioxidant, a viscosity modifier, and/or the like which are pharmaceutically acceptable. Further, a malaria therapeutic agent such as quinine, chloroquine, mefloquine, or an artemisinin derivative may be added, as needed, as a constituent of the pharmaceutical composition so as to constitute a complex drug.

The pharmaceutically acceptable carrier is not limited to a specific one, and preferably has properties of not hindering a function (treatment of malaria) of the Ca transport inhibitor when administered concurrently with the Ca transport inhibitor and causing no substantial adverse effect on a human or an animal to which the therapeutic agent is to be administered.

The carrier can be any of a wide variety of carriers conventionally known in the art. Specific example of the carrier encompass, but are not limited to, water, a variety of salt solutions, alcohol, plant oil, polyethylene glycol, gelatin, lactose, amylose, magnesium stearate, talc, silicic acid, paraffin, fatty acid monoglyceride, fatty acid diglyceride, hydroxymethyl cellulose, and polyvinyl pyrrolidone. The kind of the carrier can be appropriately selected in accordance with a form in which the pharmaceutical composition is prepared, a method by which the pharmaceutical composition is administered, etc.

The form in which the pharmaceutical composition is prepared is not limited to a specific one, and can be, for example, a tablet, a pill, powder, a solution, a suspension, an emulsion, a granules, a capsule, a suppository, or an injection. Preferably, the pharmaceutical composition is prepared in an injection or in a formulation for oral administration. For example, from the viewpoint of portability, easiness of administration, etc., a form for oral administration such as a tablet is preferable. From the viewpoint of easiness in controlling, at a predetermined timing, a blood concentration of the Ca transport inhibitor to be within a predetermined range, an injection is preferable.

The malaria therapeutic agent of the present invention can be a gene therapy agent. A specific example of the gene therapy agent is one which contains, as an active ingredient for treatment, at least one of 1) a nucleic acid which encodes a peptide having a binding activity specific to inositol trisphosphate and 2) a nucleic acid which encodes a peptide having a binding activity specific to melatonin or a homolog thereof.

The gene therapy agent may be in such a form that a nucleic acid, which is the active ingredient for treatment, is administered directly to a human or an animal by injection or may be in such a form that a vector into which a nucleic acid, which is the active ingredient for treatment, is incorporated is administered directly to a human or an animal by injection. The vector is not limited to a specific one, and can be, for example, a vector applicable to gene therapy, such as an adenovirus vector, an adeno-associated virus vector, a herpesvirus vector, a vaccinia virus vector, or a retrovirus vector. Note that the gene therapy agent may be a liposomal formulation.

The vector constituting the gene therapy agent preferably has an expression regulatory sequence incorporated therein for causing a nucleic acid encoding the aforementioned peptide to be specifically expressed in a malaria parasite. The expression regulatory sequence is, for example, a promoter or an enhancer, more specifically, a promoter sequence of calmodulin derived from malaria, a promoter sequence of a heat shock protein 86 (HSP86), or the like.

What is meant by “causing a nucleic acid to be specifically expressed in a malaria parasite” is a state in which the nucleic acid is not substantially expressed in a human or an animal to be treated, but is expressed only in the malaria parasite. This allows an effect of the gene therapy agent to selectively act on the malaria parasite.

The descriptions in [1. Method for Treating Malaria] can be referred to as for the other matters concerning the therapeutic agent of the present invention.

[4. Method for Screening for Candidate for Malaria Therapeutic Agent]

A method (1) of the present invention for screening for a candidate for a malaria therapeutic agent includes: a first step of carrying out in vitro synchronized-culturing of a malaria parasite and adding a drug to be screened to the malaria parasite while a developmental stage of the malaria parasite is being between a ring form stage and an early schizont stage; and a second step of selecting the drug as a candidate for the malaria therapeutic agent in a case where the addition of the drug suppresses development of the malaria parasite or kills the malaria parasite.

Note that the first step of adding the drug to be screened is preferably carried out while a developmental stage of the malaria parasite is being between a ring form stage and a trophozoite stage, more preferably in an early ring form stage or a trophozoite stage, especially preferably in a trophozoite stage.

The method (1) is based on a finding “administering an agent that can suppress calcium oscillation with which both melatonin and inositol trisphosphate are involved while a malaria parasite is being between a ring form stage and an early schizont stage kills the malaria parasite”. This allows screening for a candidate for a therapeutic agent that exhibits an action mechanism different from a conventional one.

A method for carrying out the synchronized-culturing in the first step and a method for checking the developmental stage of the malaria parasite are not limited in particular, and can be, for example, methods employed in Examples that will be described later.

A method (2) of the present invention for screening for a candidate for a malaria therapeutic agent includes: a first step of adding a drug to be screened to a malaria parasite that is being cultured in vitro; a second step of measuring a first amount of calcium ions exited from an intracellular organelle of the malaria parasite to an outside of the intracellular organelle and/or a second amount of calcium ions entered from an outside of a cell of the malaria parasite into the cell; and a third step of selecting the drug as the candidate for the malaria therapeutic agent in a case where the addition of the drug reduces the first amount and/or the second amount.

The method (2) is based on a finding “administering an agent that can suppress calcium oscillation with which both melatonin and inositol trisphosphate are involved kills the malaria parasite”. This allows screening for a candidate for a therapeutic agent that exhibits an action mechanism different from a conventional one.

Note that a method for measuring the first amount and the second amount of calcium ions in the second step is not limited in particular, and can be, for example, a method employed in Examples that will be described later.

Each of the methods (1) and (2) can also be considered as a method for screening for a candidate for an agent for killing a malaria parasite.

[5. Modes of Method of Present Invention for Treating Malaria]

In the method of the present invention for treating malaria, it is preferable that the drug is for suppressing calcium ion exit from an endoplasmic reticulum as the intracellular organelle to an outside of the endoplasmic reticulum.

In the method of the present invention for treating malaria, it is preferable that the drug is for inhibiting calcium ion exit from an intracellular organelle of a malaria parasite in a ring form and/or a trophozoite to an outside of the intracellular organelle and/or calcium ion entry from an outside of a cell of the malaria parasite into the cell.

In the method of the present invention for treating malaria, it is preferable that the drug contains an inhibitor of melatonin, an inhibitor of a melatonin homolog in the malaria parasite, an inhibitor of a melatonin receptor, or an inhibitor of a melatonin receptor homolog in the malaria parasite.

In the method of the present invention for treating malaria, it is preferable that the drug contains an inhibitor of an inositol trisphosphate receptor or an inhibitor of an inositol trisphosphate receptor homolog in the malaria parasite.

In the method of the present invention for treating malaria, it is preferable that the drug contains a compound or a peptide that has a binding activity specific to inositol trisphosphate, or a nucleic acid encoding the peptide. It is more preferable that the drug contains, as the peptide, the peptide represented by SEQ ID NO: 1. The drug may contain a peptide that has 80% or more sequence homology, preferably 90% or more sequence homology, especially preferably 95% or more sequence homology with a peptide represented by SEQ ID NO: 1, provided that a function of having a binding activity specific to inositol trisphosphate is not impaired. A person skilled in the art can appropriately use, as the peptide contained in the drug, a peptide that is encoded by a nucleotide sequence that hybridizes, under a stringent condition, to a sequence complementary to a nucleotide sequence encoding the aforementioned peptide or to a probe that can be prepared from the nucleotide sequence encoding the aforementioned peptide. The stringent condition is, for example, washing once, preferably two to three times at 60° C. under a salt concentration corresponding to a 1×SSC buffer containing 0.1% SDS, preferably a 0.1×SSC buffer containing 0.1% SDS. It is further preferable that the drug contains a vector containing (i) the nucleic acid encoding the peptide and (ii) an expression regulatory sequence that is linked with the nucleic acid and that causes the nucleic acid to be specifically expressed in the malaria parasite.

In the method of the present invention for treating malaria, it is preferable that a timing at which the drug is administered is determined in accordance with a developmental stage of the malaria parasite in the human or the animal. It is more preferable that the timing at which the drug is administered is determined so that a blood concentration of the drug reaches the therapeutically effective amount while the developmental stage of the malaria parasite in the human or the animal is being between a ring form stage and an early schizont stage, further more preferably between the ring form stage and a trophozoite stage, especially preferably in an early ring form stage or the trophozoite stage, most preferably in the trophozoite stage.

### EXAMPLES

The present invention is more specifically described with reference to Examples etc. below, but is not limited to these.

[Materials and Methods]

Materials and methods common to Examples are explained first below.

(1) Culturing of Falciparum Malaria Parasite (P. falciparum)

An FCR-3 strain of falciparum malaria parasite (P. falciparum) (reference document: Hatabu T, Takada T, Taguchi N, Suzuki M, Sato K, Kano S. (2005) Antimicrob Agents Chemother. 2005 February; 49(2):493-6.) was cultured in RPMI culture medium (Invitrogen/GIBCO) in accordance with the method of Trager and Jensen (reference document: Trager, W. 86 Jensen, J. B. (1976) Science 193, 673-675.). The FCR-3 strain is a strain which takes an approximately 40 hours to undergo one development/proliferation cycle of invasion of merozoites into erythrocytes, formation of ring forms, formation of trophozoites, formation of schizonts, release of mature merozoites, and invasion of the mature merozoites into erythrocytes. To the RPMI culture medium, 0.5 weight % AlubumaxI (Invitrogen), 25 mM HEPES, 24 mM sodium hydrogen carbonate, 0.5 g/L L-glutamine, 50 mg/L hypoxanthine, 25 μg/ml gentamicin (Sigma), and human erythrocytes with 5% hematocrit (provided by a healthy Japanese volunteer) were added. Further, synchronized culturing was carried out with the use of 5 weight % d-sorbitol (reference document: Lambros, C. 86 Vanderberg, J. P. (1979) J. Parasitol. 65, 418-420.) to synchronize developments of P. falciparum.

(2) Assay for Examining Inhibition of Development of P. falciparum

Effects of 2-APB (2-aminoethyl diphenylborinate) and LZ (luzindole) on intraerythrocytic falciparum malaria parasite development were examined with the use of a culture of falciparum malaria parasites in a ring form stage with initial parasitaemia of approximately 1%.

First, 500 μl of the culture was injected into each well of a tissue culture plate (24-well flat-bottom plate, Corning). 2-APB and LZ were dissolved in dimethyl sulfoxide (DMSO, HYBRI-MAX (Registered Trademark), Sigma) to be 10 mM and 100 mM in concentration, respectively. In this way, stock solutions of these compounds were prepared. The stock solutions were diluted with the RPMI culture medium, and were added to each well of the tissue culture plate in an amount to be a predetermined final concentration in the well. Further, DMSO was diluted with the PPMI culture medium and was added to each well of a tissue culture plate in an amount to be a predetermined final concentration in the well, and was used as a control. Culturing was carried out in each well for a predetermined period of time.

After elapse of the predetermined period of time, a drop of the erythrocytes-containing culture in each well of the tissue culture plate was smeared on a glass slide and giemsa-stained. The number of erythrocytes infected with falciparum malaria parasites out of 2000 erythrocytes was counted so as to determine parasitaemia.

(3) Intracellular Fluorescence Ca²⁺ Imaging of P. falciparum in Intraerythrocytic Stage and Data Analysis (Corresponding to Example 1)

0.5 ml of the culture of infected erythrocytes (5×10⁸ erythrocytes/ml) was diluted 10-fold with BSA(−) culture medium for Ca²⁺ imaging (RPMI 1640 culture medium without phenol red (Invitrogen/Gibco) supplemented with 25 mM HEPES, 24 mM sodium acid carbonate, 0.5 g/L L-glutamine and 50 mg/L hypoxanthine). Next, 1 ml of the culture of the erythrocytes that had been thus diluted was collected by centrifugation (at 1,000 g for five minutes at room temperature), then resuspended in 350 μL BSA(−) culture medium constituted by the same constituents as the BSA(−) culture medium mentioned above (hereinafter referred to as “erythrocyte resuspension (1)”).

A loading solution was prepared for loading of a Ca²⁺ fluorescence indicator (fluo4-AM) and staining of nuclei with Hoechst 33342, and fluo4-AM (BSA(−) culture medium containing 0.1 mg/ml Hoechst 33342 (Dojindo), 100 μM fluo4-AM (Invitrogen/Molecular Probes), and 100-fold dilution of PowerLoad (Invitrogen) was loaded. With 150 μL of this loading solution, 350 μL of the erythrocyte resuspension (1) was mixed, and the mixture was shaken at 200 rpm for 30 to 60 minutes at 37° C.

Erythrocytes were then washed once with 10 mL BSA(−) culture medium (1000 g for 5 minutes at room temperature) and resuspended in 600 μL BSA(+) culture medium (the BSA(−) culture medium supplemented with 0.5 weight % Albumax 1 and 25 μg/mL gentamicin (Sigma)) (hereinafter referred to as “erythrocyte resuspension (2)”). Next, 100 μL of the erythrocyte resuspension (2) was inoculated in a 35 mm glass-bottomed dish (MatTek Corp.) coated with 0.1 mg/ml poly-L-lysine. After 30 minutes of incubation in an O₂/CO₂ incubator, suspended erythrocytes were gently washed with BSA(+) culture medium before collecting.

The glass-bottomed dish was then placed in a culture chamber in which an O₂ level, a CO₂ level, temperature, and humidity were maintained under conditions (O₂ level: 5%, CO₂ level, 5%, temperature: 37° C.) used for conventional in vitro culturing of malaria parasites.

Sequential time-lapse imaging of Hoechst 33342 and fluo4-AM and acquisition of transparent images were carried out with the use of a Leica confocal electron microscope system (Leica TCS SP5 II, Leica Microsystems) with a 63× (N.A. 1.42) oil immersion objective lens. Hoechst 33342 and fluo4-AM were excited at an excitation wavelength 410 nm (with the use of a diode laser) and an excitation wavelength 488 nm (with the use of an argon laser), respectively. The acquisition of the transparent images and acquisition of light emissions were carried out by the true spectral detection method developed by Leica Microsystems. The imaging was carried out at intervals of every 5 to 15 seconds for duration of 300 to 600 seconds. A fluorescence intensity of fluo4-AM was calculated by subtraction of background fluorescence (F) and normalized to minimum fluorescence during the imaging period (Fₘᵢₙ).

(4) Method of Statistical Analysis

Differences among assays were evaluated by a Student's t-test. Among p values obtained by the test, p values less than 0.05 (p<0.05) were considered as being statistically significant.

(5) Malaria Parasite Size Measurement and Electron Microscopy

Giemsa-stained smears were observed by Nikon Eclipse 80i microscope (Nikon), photographed by Nikon DXM 1200F camera (Nikon) and uploaded on a personal computer by digital photograph manager software (ACT-1; Nikon). In order to measure falciparum malaria parasite size, 50 falciparum malaria parasites were randomly selected, and areas containing these falciparum malaria parasites were manually delineated with lines on a screen. Area, perimeter, and maximum diameter of the falciparum malaria parasites cells was calculated by WinROOF software package Ver.5.8.1 (Mitani, Japan).

(6) Transmission Electron Microscopy

Transmission electron microscopy was carried out in accordance with a published report (reference document: Kawai, S., Kano, S., Chang, C. 86 Suzuki, M. The effects of pyronaridine on the morphology of falciparum malaria parasites in Aotus trivirgatus. Am. J. Trop. Med. Hyg. 55, 223-229 (1996)). Specimens to be subjected to the electron microscopy were fixed for approximately 2 hours in 2.5% (V/V) glutaraldehyde buffered with 0.1 M phosphate buffer (pH 7.4, at 4° C.). The specimens were then post-fixed in 1% (w/V) osmium tetroxide for 1 hour. The specimens thus fixed were dehydrated in ascending concentrations of ethanol followed by 15 minutes of treatment with propylene oxide, and embedded in an Epon 812 resin. Block of the Epon 812 resin thus obtained were cut with the use of an ultramicrotome (Porter-Blim MT-2; Ivan Sorvall) having a diamond knife (Diatome). Sections thus obtained were mounted on 200-mesh copper grids, stained with uranyl acetate and lead citrate, and observed with the use of a JEOL JEM-1011 transmission electron microscope.

(7) Staining of Nucleus and Endoplasmic Reticulum

Nuclei and endoplasmic reticulums of the falciparum malaria parasites were stained with Hoechst 33342 and

ER-Tracker Red (Invitrogen). The staining with Hoechst 33342 was carried out as described above for the purpose of fluorescence Ca²⁺ imaging. ER-Tracker Red was then added to the erythrocyte suspension so as to be 0.5 μM in final concentration, and the mixture was shaken at 200 rpm for 30 minutes at 37° C.

### Example 1

(1) Spontaneous Ca²⁺ Oscillations in Falciparum Malaria Parasites and Inhibition of Ca²⁺ Oscillations by 2-APB

FIGS. 1 and 2 show results of observation of Ca²⁺ oscillations and results of Ca²⁺ oscillation inhibition experiments.

(b) and (f) of FIG. 1 are graphs showing results of observing, by fluorescence Ca²⁺ imaging, the behavior of Ca²⁺ in cytoplasms of falciparum malaria parasites (P. falciparum) which were cultured after addition of 100 μM DMSO serving as a control in an early ring form (ER) stage and in a trophozoite (T) stage, respectively.

(c) and (g) of FIG. 1 are graphs showing results of observing, by fluorescence Ca²⁺ imaging, the behavior of Ca²⁺ in cytoplasms of falciparum malaria parasites (P. falciparum) which were cultured after addition of 100 μM 2-APB in an early ring form (ER) stage and in a trophozoite (T) stage, respectively. 2-APB (2-aminoethyl diphenylborinate) was developed and established by the inventors of the present invention as an inhibitor of an inositol 1,4,5-trisphosphate receptor Ca²⁺ channel.

(d), (h), and (j) of FIG. 1 are graphs showing results of observing, by fluorescence Ca²⁺ imaging, the behavior of Ca²⁺ in cytoplasms of falciparum malaria parasites (P. falciparum) which were cultured after addition of 100 μM DMSO serving as a control in a late ring form (LR) stage, a schizont (S) stage, and a merozoite (M) stage, respectively.

(e), (i), and (k) of FIG. 1 are graphs showing results of observing, by fluorescence Ca²⁺ imaging, the behavior of Ca²⁺ in cytoplasms of falciparum malaria parasites (P. falciparum) which were cultured after addition of 100 μM 2-APB in a late ring form (LR) stage, a schizont (S) stage, and a merozoite (M) stage, respectively.

In (b) through (k) of FIG. 1, dots shown in different forms indicate results obtained for respective different malaria parasites. In (b), (d), (f), (h), and (j) of FIG. 1, the arrow heads indicate images of the ER (early ring form), LR (late ring form), T (trophozoite), S (schizont), and M (merozoite) in an erythrocyte, respectively, and each of the scale bars indicates 5 μm.

As shown in (b) and (f) of FIG. 1, each of the controls in the early ring form stage and the trophozoite stage exhibited spontaneous Ca²⁺ oscillations. The frequency of Ca²⁺ oscillations was higher in early ring forms than that in trophozoites. Note that what is meant by “early ring form” is a malaria parasite that has a cell size smaller than a trophozoite, that has a hemozoine formed in its cytoplasm, and that has a single nucleus, and what is meant by “trophozoite” is a malaria parasite with a single nucleus.

In contrast, as shown in (c) and (g) of FIG. 1, treatment of malaria parasites with 100 μM 2-APB almost completely blocked Ca²⁺ oscillations, which were observed in the controls.

On the other hand, as shown in (d), (h), and (j) of FIG. 1, in the late ring forms (LR), schizonts (S) and merozoites (M), relatively very small Ca²⁺ fluctuations were observed, and treatment with 100 μM 2-APB brought about no notable effect (see (e), (i), and (k) of FIG. 1). Note that what is meant by “late ring form” is a parasite that has a cell size ranging between a cell size of an early ring form and a cell size of a trophozoite and that has no hemozoine.

Next, quantitative analysis was carried out on the effect of 2-APB on the amplitude of periodic Ca²⁺ fluctuations in the late ring form (LR) stage, the schizont (S) stage, and the merozoite (M) stage in which very small Ca²⁺ fluctuations were observed. A mean amplitude was calculated by subtracting a mean minimal value of F/Fₘᵢₙ from a mean maximal value of F/Fₘᵢₙ. As a result, a statistically significant effect of 2-APB was observed only in the merozoite stage (see (1) of FIG. 1).

(2) Confirmatory Experiment Using U73122, Tg, and CMA

As shown in FIG. 1, addition of 2-APB in the early ring forms and trophozoites inhibited spontaneous Ca²⁺ oscillations. This fact suggests that Ca²⁺ oscillations are regulated by an inositol trisphosphate receptor Ca²⁺ channel that is activated by inositol trisphosphate binding.

In order to confirm this suggestion, a confirmatory experiment using U73122, which is a generally used phospholipase C inhibitor, was carried out first. In a malaria parasite, a phospholipase C signal transduction pathway is suggested to be involved in release of Ca²⁺ from a Ca²⁺ store in the malaria parasite (Reference Document: Hotta, C. T., Markus, R. P. 86 Garcia, C. R. Braz J Med Biol Res 36, 1583-1587 (2003).).

(a) and (b) of FIG. 2 are graphs showing results of fluorescence Ca²⁺ imaging of falciparum malaria parasites in an early ring form (ER) stage and a trophozoites (T) stage, respectively, after 5 minutes of pre-treatment with 10 μM U73122. As shown in (a) and (b) of FIG. 2, the pre-treatment almost completely blocked Ca²⁺ oscillations both in the early ring form (ER) stage and the trophozoite (T) stage.

In order to investigate a source of Ca²⁺ which is related to Ca²⁺ oscillations, pre-treatment for artificially releasing Ca²⁺ with the use of thapsigargin (Tg) or concanamycin A (CMA) was carried out before fluorescence Ca²⁺ imaging. Details are described below.

In apicomplexan parasites, two intracellular Ca²⁺ stores, i.e., endoplasmic reticulum and acidocalcisomes are known to involve in Ca²⁺ release. In order to selectively release Ca²⁺ from any one of the endoplasmic reticulum and acidocalcisomes, falciparum malaria parasites were pre-treated with the use of thapsigargin (Tg), which is a specific inhibitor of sarco/endoplasmic reticulum Ca²⁺-ATPase, and concanamycin A (CMA), which is a specific inhibitor of vacuolar-type H⁺-ATPase, respectively.

The effect of these compounds on an increase of Ca²⁺ leakage was confirmed by Ca²⁺ imaging both in early ring forms (white circles) and trophozoites (black rectangles) with the use of perfusion experiments. The result is shown in (a) and (b) of FIG. 3. As shown in (c) and (d) of FIG. 2, Ca²⁺ release from the endoplasmic reticulum by pre-treatment with 2 μM Tg for 30 minutes blocked Ca²⁺ oscillations in early ring forms (ER) and trophozoites (T). In contrast, as shown in (e) and (f) of FIG. 3, Ca²⁺ release from acidocalcisomes by pre-treatment with 100 nM CMA had no effect on Ca²⁺ oscillations in early ring forms (ER) and trophozoites (T).

These results indicate that spontaneous inositol trisphosphate-induced Ca²⁺ release (IICR) from endoplasmic reticulum occurred in early ring forms and trophozoites during intraerythrocytic falciparum malaria parasite development.

### Example 2

### Falciparum Malaria Parasite Development Inhibition/Death by Treatment with 2-APB

(1) Inhibition of Intraerythrocytic Falciparum Malaria Parasite Development by 2-APB

The results of the development inhibition experiments are shown in (a) through (d) of FIG. 4. Note that (d) of FIG. 4 relates especially to the item (2) that will be described later.

(a) of FIG. 4 shows a result of culturing a falciparum malaria parasite FCR-3 strain in a 24-well tissue culture plate for 40 hours after start of synchronized culturing. Each experimental group was cultured with the use of three wells for 20 hours, 30 hours, and 40 hours before being assayed, and thin smears of erythrocytes were prepared for falciparum malaria parasite counting. In (a) and (d) of FIG. 4, parasitaemia of ring forms (R), trophozoites (T), early schizonts (ES) and late schizonts (LS) is shown as mean±SD of 3 counts of each experimental group. Stages with parasitaemia of less than 0.1% are not shown.

(b) of FIG. 4 shows morphology of intraerythrocytic falciparum malaria parasites in the cultures of (a) of FIG. 4.

As shown in (a) and (b) of FIG. 4, in the presence of 100 μM 2-APB, intraerythrocytic development of falciparum malaria parasites was clearly delayed compared with the DMSO controls. In the controls, falciparum malaria parasites had normal morphology throughout the culturing period. In contrast, falciparum malaria parasites cultured in the presence of 2-APB exhibited abnormal morphology.

Specifically, falciparum malaria parasites in the control cultures developed into early schizonts (falciparum malaria parasites with fewer than 8 nuclei) at 20 hours from the start of the synchronized culturing. These schizonts developed into healthy late schizonts (falciparum malaria parasites with at least 8 nuclei) at 30 hours from the start of the synchronized culturing. Then, mature merozoites were released, and ring forms in a next infection cycle were observed at 40 hours from the start of the synchronized culturing.

In contrast, falciparum malaria parasites cultured in the presence of 2-APB remained at the trophozoite stage (falciparum malaria parasites with a single nucleus) with abnormal morphology at 20 hours from the start of the synchronized culturing. These trophozoites could develop into early schizonts at 30 hours from the start of the synchronized culturing and into late schizonts at 40 hours from the start of the synchronized culturing, but these early schizonts and late schizonts exhibited abnormal morphology (see (b) of FIG. 4).

In order to quantitatively examine the effect of 2-APB on intraerythrocytic falciparum malaria parasite development, the area, perimeter and maximum diameter (hereinafter referred to collectively as “three parameters”) of falciparum malaria parasite cells were analyzed at 15 hours, 30 hours, and 40 hours from the start of the synchronized culturing. At 40 hours from the start of the synchronized culturing, only schizonts were analyzed. At 15 hours, 30 hours, and 40 hours from the start of the synchronized culturing, falciparum malaria parasites cultured in the presence of 100 μM 2-APB showed significant decreases in the three parameters as compared to those cultured in the presence of DMSO. This suggests that 2-APB delays intraerythrocytic falciparum malaria parasite development (see (c) of FIG. 4). Furthermore, analysis of the falciparum malaria parasite size revealed that the increases in the three parameters were terminated at 30 hours after 2-APB treatment. This suggests that the critical time of the effect of 2-APB is approximately 30 hours.

In (c) of FIG. 4, columns and error bars represent the mean±S.D. Fifty falciparum malaria parasites were measured in terms of the three parameters at each time point. P values compared with DMSO controls are given below each figure (two-tailed unpaired t test with Welch's correction).

(2) Death of Falciparum Malaria Parasites by 2-APB Treatment

In order to investigate fates of the schizonts with abnormal morphology which were obtained through culturing in the presence of 2-APB, the synchronized culturing was prolonged for another 30 hours. (d) of FIG. 4 shows results of counting of the falciparum malaria parasites at 40 hour (corresponding to the final stage of (a) of FIG. 4) and 70 hour from the start of the synchronized culturing. Culture medium containing DMSO or 2-APB was replaced with a culture medium containing no DMSO or 2-APB at 40 hour from the start of the synchronized culturing. In (d) of FIG. 4, a representative one of experimental results in the 3 wells is shown for each experimental group.

At 70 hours from the start of the culturing, falciparum malaria parasites cultured with the controls (DMSO) developed into late schizonts (some of the falciparum malaria parasites developed into ring forms). Meanwhile, at 70 hours from the start of the culturing, falciparum malaria parasites cultured in the presence of 2-APB developed into late schizonts or ring forms with abnormal morphology (1 in 5000 to 8000 erythrocytes). Further, erythrocyte parasitaemia of the falciparum malaria parasites cultured in the presence of 2-APB gradually decreased with passage of time, and reached substantially zero (the falciparum malaria parasites were killed) at 70 hours from the start of the synchronized culturing.

As shown in FIG. 5, at 20 hours from the start of the synchronized culturing ((a) of FIG. 5) and 40 hours from the start of the synchronized culturing ((b) of FIG. 5), the falciparum malaria parasites could develop normally in erythrocytes treated with 2-APB only in advance, similar to those in DMSO-pretreated erythrocytes. This suggests that the effect of 2-APB is not due to the disruption of erythrocyte physiology. These results suggest that 2-APB inhibits intraerythrocytic falciparum malaria parasite development by blocking a normal cell cycle of falciparum malaria parasites, resulting in death of the falciparum malaria parasites.

(3) Effect of 2-APB Treatment on Chloroquine-Resistant Strain of Falciparum Malaria Parasites

An effect of 2-APB on intraerythrocytic development of a chloroquine-resistant strain K1 of falciparum malaria parasites (P. falciparum) was examined by synchronized culturing malaria parasites in ring forms with initial parasitaemia of approximately 2%. The culturing was terminated at 24 hours, 48 hours, and 72 hours from the start of the synchronized culturing, and thin smears of erythrocytes were prepared for falciparum malaria parasite counting. Culture medium with 100 μM DMSO or 100 μM 2-APB was replaced at 24 hours and 48 hours from the start of the synchronized culturing. (e) of FIG. 4 shows representative results of 3 independent experiments. Parasitaemia of ring forms (Rf), trophozoites (T), early schizonts (ES) and late schizonts (LS) is shown as mean±S.D. of 3 wells. Stages with parasitaemia of less than 0.1% are not shown. At 24 hours from the start of the assay, a tendency towards decreasing parasitaemia of falciparum malaria parasite in trophozoites was reproducibly observed in a system cultured in the presence of 100 μM 2-APB (see (e) of FIG. 4). The inhibitory effect of 2-APB at 24 hours from the start of the assay was confirmed by measuring the area, perimeter and maximum diameter of the falciparum malaria parasite cells (see FIG. 10). At 48 hours from the start of the assay, intraerythrocytic malaria parasite development in the presence 2-APB was delayed compared to that in the presence of DMSO, similar to that observed in the FCR3 strain. As a result of further 24 hours of the assay (at 72 hours from the start of the assay), it was revealed that the number of erythrocytes infected with falciparum malaria parasite (in which there was a high level of parasitaemia) in the presence of 2-APB was much lesser than that in the presence of DMSO.

The effect of 2-APB on the K-1 strain at 24 hour from the start of the assay is relatively weak (see (e) of FIG. 4). It is considered that this is because a large part of the falciparum malaria parasite K-1 strain does not reach a stage in which 2-APB gave a lethal effect on the FCR-3 strain (stage between a trophozoite stage and an early schizont stage: see Example 3 and FIG. 6).

### Example 3

(1) 2-APB Inhibits Intraerythrocytic Falciparum Malaria Parasite Development at Initial Stage

Next, in order to investigate a stage in which inhibition of intraerythrocytic falciparum malaria parasite development occurs, 2-APB was added to cultures at different timings. FIG. 6 shows results of experiments about an effect of 2-APB on cultures of falciparum malaria parasites in different developmental stages. Results of two independent experiments (Ex-1 and Ex-2) are shown as representative results. In the experiments, falciparum malaria parasite FCR-3 strain was synchronized-cultured with the use of 24-well tissue culture plate. At 40 hours from the start of the synchronized culturing, the culturing was terminated, and the number of falciparum malaria parasites was counted (3 wells was used for each experimental group. Stages with parasitaemia of less than 0.1% are not shown).

(a) of FIG. 6 is a graph showing a result of culturing in which 2-APB was added at start of synchronized culturing and culture medium was replaced with culture medium containing no 2-APB at 10 hours from the start of the synchronized culturing at which falciparum malaria parasites are in ring forms. (b) of FIG. 6 is a graph showing a result of culturing in which 2-APB was added at start of synchronized culturing and culture medium was replaced with culture medium containing no 2-APB at 21 hours from the start of the synchronized culturing at which the falciparum malaria parasites developed into a transition stage from trophozoites to early schizonts. (c) of FIG. 6 is a graph showing a result of culturing in which 2-APB was added at 21 hours from start of the culturing. (d) of FIG. 6 is a graph showing a result of culturing in which 2-APB was added at 28 hours from start of the culturing at which falciparum malaria parasites developed into late schizonts. The total culture time is 45 hours. In (a) through (c) and (e) of FIG. 6, parasitaemia (%) of ring forms (R), trophozoites (T), early schizonts (ES), and late schizonts (LS) is mean±SD of three wells constituting each experimental group. Stages with parasitaemia of less than 0.1% are not shown.

Although a significant difference was observed in parasitaemia of the ring forms at 40 hours from the start of the assay both in a case where 2-APB was added at start of the assay and was removed at a ring form stage and in a case where 2-APB was added at start of the assay and was removed in a stage between trophozoite and early schizont stages, an effect of 2-APB was greater in the case where 2-APB was present until the stage between the trophozoite and early schizont stages.

In a case where 2-APB was added in the stage between the trophozoite and early schizont stages, a greater effect was observed on parasitaemia of ring forms at 40 hours from the start of the assay, as compared with a case where 2-APB was removed at the ring form stage or the stage between the trophozoite and early schizont stages. In the results of the two independent experiments (Ex-1 and Ex-2) in which 2-APB was added in the stage between the trophozoite and early schizont stages, a remarkable difference was observed in parasitaemia of ring forms at 40 hours from the start of the assay. This is estimated from (d) of FIG. 6 to be relevant with a ratio of trophozoites and schizonts present at the time of the addition of 2-APB. A comprehensive analysis of the results shown in (a) through (c) of FIG. 6 strongly suggests that exposure to 2-APB at a trophozoite stage gives a severe effect on ring form parasitaemia at 40 hours from the start of the assay (parasitaemia at start of a next developmental cycle).

On the other hand, in a case where 2-APB was added at a later timing, for example, at a late schizont stage, the falciparum malaria parasites formed ring forms, as observed in the control cultures. However, the ring form parasitaemia at 45 hours from the start of the assay significantly decreased due to the addition of 2-APB ((a) of FIG. 6: *p<0.05).

The results suggest that 2-APB inhibits maturing of late schizonts, release of merozoites into an outside of erythrocytes, and/or invasion of merozoites into erythrocytes.

Combined with the results of the Ca²⁺ imaging experiments, these results demonstrated for the first time that inositol trisphosphate-induced spontaneous Ca²⁺ oscillations in a trophozoite stage are extremely important for intraerythrocytic falciparum malaria parasite development. Further, it was concluded that inositol trisphosphate-induced spontaneous Ca²⁺ periodic fluctuations in the merozoite stage play an important role in invasion of falciparum malaria parasites into erythrocytes.

In order to investigate an effect of 2-APB in the ring form stage, 100 μM 2-APB was added to falciparum malaria parasites that were being synchronized-cultured, and sizes of the falciparum malaria parasites were measured after 10 hours of culturing and after 20 hours of culturing. In some assays (“removed at 10 h” in (f) of FIG. 6), 100 μM 2-APB was added at start of the culturing and removed after 10 hours of culturing, and then sizes of falciparum malaria parasites were measured after 20 hours of culturing (see (f) of FIG. 6). Similar experiments were carried out also on falciparum malaria parasites cultured in the presence of 100 μM DMSO which served as a control. Fifty falciparum malaria parasites were measured in each experimental group. P values compared with the controls (in the presence of DMSO) are given in each panel (two-tailed unpaired t test with Welch's correction).

In the falciparum malaria parasites cultured in the presence of 100 μM DMSO which served as a control, increases in area, perimeter and maximum diameter at 20 hours from the start of the assay were higher than those observed at 10 hours from the start of the assay. A similar result was obtained in a system in which DMSO was removed at 10 hours from the start of the assay. In contrast, in the falciparum malaria parasites cultured in the presence of 100 μM 2-APB for 10 hours and 20 hours from the start of the assay, these three parameters were significantly smaller than those in the falciparum malaria parasites cultured in the presence of DMSO. However, in the case where 2-APB was removed at 10 hours from the start of the assay, all of the three parameters were just slightly smaller than those in the falciparum malaria parasites cultured in the presence of DMSO, but recovered to a level at which no statistically significant difference was detected.

Considering the reversible effect of 2-APB in the early ring form stage (see (f) of FIG. 6), it is estimated that the lethal effect of 2-APB on intraerythrocytic falciparum malaria parasite development was caused mainly by the blockage of Ca²⁺ oscillations in the trophozoite stage.

### Example 4

(1) Inhibition of Intraerythrocytic Falciparum Malaria Parasite Development by LZ

(a) through (c) of FIG. 7 show results of experiments on development inhibition in the presence of 250 μM LZ. Note that a LZ concentration was determined on the basis of a published report (reference document: Beraldo F. H., Mikoshiba K. 86 Garcia C. R. (2007) J. Pineal. Res. 43, 360-364.). Culture experiments in the presence of DMSO were used as controls. FIG. 7 shows results of culturing falciparum malaria parasite FCR-3 strain in a 24-well tissue culture plate for 70 hours from start of synchronized culturing. The culturing was carried out with the use of three wells for each experimental group, and terminated at 20 hours ((a) of FIG. 7), 40 hours ((b) of FIG. 7), and 70 hours ((c) of FIG. 7) from start of the culturing, and subjected to assay. Thin smears of erythrocytes were prepared for falciparum malaria parasite counting. In FIG. 7, parasitaemia (%) of ring forms (R), trophozoites (T), early schizonts (ES) and late schizonts (LS) is shown as mean±SD of 3 wells constituting each experimental group. Stages with parasitaemia of less than 0.1% are not shown.

As shown in FIG. 7, intraerythrocytic falciparum malaria parasite development was clearly inhibited in the presence of 250 μM LZ.

Specifically, the falciparum malaria parasites in the control culture developed into late trophozoites and early schizonts at 20 hours from the start of the assay. These falciparum malaria parasites developed into healthy early and late schizonts and into a transition stage from ring forms to early trophozoites in a next developmental cycle at 40 hours from the start of the assay. These falciparum malaria parasites developed into healthy trophozoites and early and late schizonts and into ring forms in the next developmental cycle at 70 hours from the start of the assay.

In contrast, the falciparum malaria parasites cultured in the presence of LZ remained in a transition stage from ring forms to early trophozoites at 20 hours from the start of the assay. Some of the ring forms/early trophozoites could develop into early schizonts. However, most of the falciparum malaria parasites remained in the transition stage from ring forms to early trophozoites even at 40 hours from the start of the assay. These falciparum malaria parasites remained in the transition stage from ring forms to early trophozoites even at 70 hours from the start of the assay. The early schizonts observed at 40 hours from the start of the assay were estimated to have stopped developing and died.

The results suggest that LZ inhibits intraerythrocytic falciparum malaria parasite development by blocking a normal cell cycle of falciparum malaria parasites. Note that LZ is an antagonist of a melatonin receptor.

### Example 5

### Severe Malaria Parasite Degeneration Caused by 2-APB

Degeneration of an ultrafine structure caused by 2-APB was observed with the use of a transmission electron microscope. As shown in (a) of FIG. 8, the falciparum malaria parasites cultured in the presence of DMSO maintained a normal structure at 30 hours from start of an assay. In contrast, in falciparum malaria parasites cultured in the presence of 100 μM 2-APB, highly dense chromatin masses in the nucleus and highly dense degeneration were observed at 30 hours from the start of the assay (see (b) and (c) of FIG. 8). The formation of Maurer's cleft and malaria pigment in food vacuoles suggests that degeneration induced by 2-APB occurred after intraerythrocytic development progressed to some extent (see (b) of FIG. 8).

In Plasmodium species, a nuclear envelope is considered as a main endoplasmic reticulum (ER) compartment. As shown in FIG. 9, it was confirmed that, in a case where falciparum malaria parasites were cultured in the presence of DMSO, endoplasmic reticulum tracker signals (ER-Tracker signals) stained blue with Hoechst 33342 surrounded the nuclei of falciparum malaria parasites, whereas in a case where falciparum malaria parasites were cultured in the presence of 2-APB, the endoplasmic reticulum tracker signals became broad and extended to the cytosol. Similarly, in electron micrographs, falciparum malaria parasites cultured in the presence of 2-APB showed a dilated nuclear envelope and endoplasmic reticulum (see (b) and (c) of FIG. 8) and the dilated nuclear envelopes connected with the dilated endoplasmic reticulum formed a reticular endoplasmic reticulum (reticular ER) structure (see (d) of FIG. 8 and FIG. 9). In a published report (reference document: Bannister, L. H., Hopkins, J. M., Fowler, R. E., Krishna, S. 86 Mitchell, G. H. Ultrastructure of rhoptry development in falciparum malaria parasite erythrocytic schizonts. Parasitology 121 (Pt 3), 273-287 (2000).) concerning observation of malaria parasites with the use of an electron microscope, it is reported that a nucleus surrounded by rough endoplasmic reticulum in a late schizont has a nuclear envelope bearing numerous ribosomal granules. Similar electron micrographs were obtained with falciparum malaria parasites cultured in the presence of DMSO (see FIG. 11). Further, falciparum malaria parasites cultured in the presence of 2-APB frequently exhibited an increased number of ribosomal granules, distributed in a line along the dilated nuclear envelope (see (e) of FIG. 8).

Although most of the falciparum malaria parasites cultured in the presence of 2-APB showed severe degeneration (see (b) and (c) of FIG. 8), schizonts in which merozoites with normal micro-organelles were formed were also present (see (f) of FIG. 8). At 40 hours from the start of the assay, the number of merozoites formed in each schizont of falciparum malaria parasites cultured in the presence of 2-APB was significantly smaller than that in the presence of DMSO (see (a) and (b) of FIG. 12; two-tailed unpaired t test with Welch's correction was carried out; (b) of FIG. 12 is raw data of the frequency distribution shown in (a) of FIG. 12). This suggests that even development of falciparum malaria parasite in which merozoites were formed normally was inhibited by 2-APB.

The present invention is not limited to the description of the embodiments and the examples above, but may be altered by a skilled person within the scope of the claims. An embodiment based on a proper combination of technical means disclosed in different embodiments is encompassed in the technical scope of the present invention.

### INDUSTRIAL APPLICABILITY

The present invention can provide a method for treating malaria, a method for killing a malaria parasite, each of which utilizes a mechanism different from a conventional one, and use of the methods.

1. A method for treating malaria, comprising the step of administering, to a human or an animal, a therapeutically effective amount of drug for suppressing calcium ion exit from an intracellular organelle of a malaria parasite to an outside of the intracellular organelle and/or calcium ion entry from an outside of a cell of the malaria parasite into the cell.

2. The method according to claim 1, wherein the drug is for suppressing calcium ion exit from an endoplasmic reticulum as the intracellular organelle to an outside of the endoplasmic reticulum.

3. The method according to claim 1, wherein the drug is for suppressing the calcium ion exit in a malaria parasite in a ring form and/or a trophozoite.

4. The method according to claim 1, wherein the drug contains an inhibitor of melatonin, an inhibitor of a melatonin homolog in the malaria parasite, an inhibitor of a melatonin receptor, or an inhibitor of a melatonin receptor homolog in the malaria parasite.

5. The method according to claim 1, wherein the drug contains an inhibitor of an inositol trisphosphate receptor or an inhibitor of an inositol trisphosphate receptor homolog in the malaria parasite.

6. The method according to claim 1, wherein the drug contains a compound or a peptide that has a binding activity specific to inositol trisphosphate, or a nucleic acid encoding the peptide.

7. The method according to claim 6, wherein the drug contains, as the peptide, the peptide represented by SEQ ID NO: 1.

8. The method according to claim 6, wherein the drug includes a vector containing (i) the nucleic acid encoding the peptide and (ii) an expression regulatory sequence that is linked with the nucleic acid and that causes the nucleic acid to be specifically expressed in the malaria parasite.

9. The method according to claim 1, wherein the drug is administered, as preventative treatment, to the human or the animal before infection with the malaria parasite.

10. The method according to claim 1, wherein a timing at which the drug is administered is determined in accordance with a developmental stage of the malaria parasite in the human or the animal.

11. The method according to claim 10, wherein the timing at which the drug is administered is determined so that a blood concentration of the drug reaches the therapeutically effective amount while the developmental stage of the malaria parasite in the human or the animal is being between a ring form stage and an early schizont stage.

12. A method for killing a malaria parasite, comprising the step of supplying, to a malaria parasite, an effective amount of drug for suppressing calcium ion exit from an intracellular organelle of the malaria parasite to an outside of the intracellular organelle and/or calcium ion entry from an outside of a cell of the malaria parasite into the cell.

13. A malaria therapeutic agent, comprising a drug for suppressing calcium ion exit from an intracellular organelle of a malaria parasite to an outside of the intracellular organelle and/or calcium ion entry from an outside of a cell of the malaria parasite into the cell.

14. A method for screening for a candidate for a malaria therapeutic agent, comprising:
the first step of synchronized-culturing a malaria parasite in vitro and adding a drug to be screened while a developmental stage of the malaria parasite is being between a ring form stage and an early schizont stage; and
the second step of selecting the drug as the candidate for the malaria therapeutic agent in a case where the addition of the drug suppresses development of the malaria parasite or kills the malaria parasite.

15. A method for screening for a candidate for a malaria therapeutic agent, comprising:
the first step of adding a drug to be screened to a malaria parasite that is being cultured in vitro;
the second step of measuring a first amount of calcium ions exited from an intracellular organelle of the malaria parasite to an outside of the intracellular organelle and/or a second amount of calcium ions entered from an outside of a cell of the malaria parasite into the cell; and
the third step of selecting the drug as the candidate for the malaria therapeutic agent in a case where the addition of the drug reduces the first amount and/or the second amount.

16. A method for preventing secondary infection with malaria, comprising the step of supplying, to blood that is outside a human or animal body and that is infected with a malaria parasite or has a risk of infection with the malaria parasite, a drug for suppressing calcium ion exit from an intracellular organelle of the malaria parasite to an outside of the intracellular organelle and/or calcium ion entry from an outside of a cell of the malaria parasite into the cell.
